(19) United States
(12) Patent Application Publication
(10) Pub. No.: US 2024/0306992 A1
(43) Pub. Date: Sep. 19, 2024

(54) WEARABLE HEALTH MONITORING DEVICE

(71) Applicant: KNOW LABS, INC., Seattle, WA (US)

(72) Inventor: JOHN CRONIN, Seattle, WA (US)

(21) Appl. No.: 18/608,461

(22) Filed: Mar. 18, 2024

Related U.S. Application Data
(60) Provisional application No. 63/490,645, filed on Mar. 16, 2023.

(51) Int. Cl. A61B 5/00; A61B 5/0205; A61B 5/022; A61B 5/05; A61B 5/145; A61B 5/256; A61B 5/28

(52) U.S. Cl. CPC A61B 5/681; A61B 5/0022; A61B 5/02055; A61B 5/02233; A61B 5/05; A61B 5/14532; A61B 5/256; A61B 5/28; A61B 5/4857; A61B 5/721

(57) ABSTRACT

A wearable health monitoring device removably attached to a smartwatch to be worn by a user in multiple configurations. The wearable health monitoring device can include a radio frequency (RF) sensing module with at least one transmit antenna and at least one receive antenna. A semiconductor chip is configured to convert low-power radio frequency signals received by at least one receive antenna into high-power radio frequency signals. A processor is communicatively coupled to an analog-to-digital converter (ADC) and configured to convert digital signals into output information. A wireless communication interface is communicatively coupled with the processor and configured to transmit the output information to the smartwatch to reflect the user's health parameters.

### CROSS-REFERENCE TO RELATED APPLICATIONS

This application claims priority to U.S. Provisional Application No. 63/490,645, filed on Mar. 16, 2023, the disclosure of which is incorporated herein by reference in its entirety.

### FIELD OF THE DISCLOSURE

The present disclosure is generally related to health monitoring device, and more particularly related to a wearable apparatus integrated with a smartwatch for real-time monitoring of health parameters.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Regular monitoring of health parameters is indispensable for modern urban and working population. Traditionally, doctors and patients alike would utilize a variety of invasive and non-invasive methods to assess vital signs including blood sugar, heart rate, and temperature. Taking a blood sample by pricking a location on a finger and determining a proportion of glucose was necessary for determining glucose levels. Although patients reliably sample their blood glucose levels with these methods, however, due to the need to puncture the skin, not everyone is willing to submit to these tests on a regular basis. In addition, there is always a risk of introducing potentially dangerous germs into a patient's body due to these intrusive procedures.

Alternatively, a non-invasive technique is used in the form of a device that is capable of accurately measuring the glucose content inside the blood of a user. The device is equipped with transmitting and receiving antennas that emit and receive radio signals into and returning from the user. The responded signals are processed for determining the glucose content. Generally, these devices are additionally integrated into wrist watches, such as, multimedia tool smartwatches or smart bands.

Moreover, some existing smart bands and watches are equipped with separate or integrated band pieces that are configured to monitor different health parameters of the user. Such additional band pieces are integrated at a back side of the smartwatches or at the straps. Further, these band pieces' act as a separate entity, and are provided with separate elements and battery. These additional band pieces may operate independently from the smartwatches and can run for longer hours due to separate battery units. However, due to fixed configuration, the accuracy of these additional band pieces may decrease. Further, these additional band pieces are configured to receive radio frequency signals, and the positioning of these additional band pieces over the strap hampers the transmission and reception of these signals. Also, these additional band pieces cannot be used during multiple activities such as playing sports, medical checkups in hospitals, etc. due to their restricted configuration.

Therefore, there is a need for an improved device having multiple configuration setup with longer battery life and uses a non-invasive technique to accurately determine multiple health parameters.

### SUMMARY

A wearable health monitoring device integrated with a smartwatch having a watch case and a watch strap. For example, a wearable health monitoring device integrated with a smartwatch having a watch case and a watch strap in which at least one transmit antenna is configured to transmit radio frequency signals underneath a skin surface of a user. At least one receive antenna is configured to receive responded radio frequency signals from the user. An analog to digital converter (ADC) is communicatively coupled with the at least one receive antenna, wherein the ADC is configured to convert the received responded radio frequency signals into digital signals. A processor is communicatively coupled to the ADC and configured to convert the digital signals into output information. A wireless communication module is communicatively coupled with the processor and configured to transmit the output information to the smartwatch to reflect a health parameter of the user. Further, the wearable health monitor is removably attached to the smartwatch

In one example, a wearable health monitoring device is removeably attachable to a smartwatch having a watch case and a watch strap. The wearable health monitoring device including at least one transmit antenna, at least one receive antenna, an analog to digital converter (ADC), a processor, a a wireless communication module, and a structure for physically interfacing with the smartwatch. The at least one transmit antenna configured to transmit radio frequency (RF) detection signals into a skin surface of a user. The at least one receive antenna configured to receive responded RF detection signals that result from the RF detection signals transmitted into the user. The analog to digital converter communicatively coupled with the at least one receive antenna, wherein the ADC is configured to convert the received responded RF signals into digital signals. The processor communicatively coupled to the ADC and configured to convert the digital signals into output information; and The wireless communication module communicatively coupled with the processor and configured to transmit the output information to the smartwatch to reflect a health parameter of the user.

In another example, a wearable health monitoring device, removeably attachable to a smartwatch having a watch case and a watch strap. The wearable health monitoring device including at least one transmit antenna, at least one receive antenna, an analog to digital converter, a processor, one or more clips, and a motion sensor. The at least one transmit antenna configured to transmit radio frequency detection signals into a skin surface of a user. The at least one receive antenna configured to receive responded RF detection signals that result from the RF detection signals transmitted into the user. The an analog to digital converter (ADC) communicatively coupled with the at least one receive antenna, wherein the ADC is configured to convert the received responded RF signals into digital signals. The processor communicatively coupled to the ADC and configured to convert the digital signals into output information. The one or more clips configured to attach over the watch strap. The motion sensor detects motion of the user during transmission of the RF detection signals by the one or more transmit antennas and during detection of the RF signals by the one or more receive antennas.

In another example, a wearable health monitoring device removably attached to a smartwatch to be worn by a user in multiple configurations. The wearable health monitoring device can include a radio frequency sensing module with at least one transmit antenna and at least one receive antenna. A semiconductor chip is configured to convert low-power radio frequency signals received by at least one receive antenna into high-power radio frequency signals. A processor is communicatively coupled to an analog-to-digital converter and configured to convert digital signals into output information. A wireless communication interface is communicatively coupled with the processor and configured to transmit the output information to the smartwatch to reflect the user's health parameters

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a wearable health monitoring device communicatively coupled to a smartwatch, according to an embodiment;

FIG. 2 illustrates a table representing derived data stored within a database, according to an embodiment;

FIG. 3 illustrates a block diagram of components of the smartwatch, according to an embodiment;

FIG. 4 illustrates the wearable health monitoring device connected to the smartwatch in a clip-on configuration, according to an embodiment;

FIG. 5 illustrates the wearable health monitoring device connected to the smartwatch in a shell around configuration, according to an embodiment; and

FIG. 6 illustrates the wearable health monitoring device connected to the smartwatch in an interposer configuration, according to an embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

US 2020/0187820 is incorporated herein by reference in its entirety.

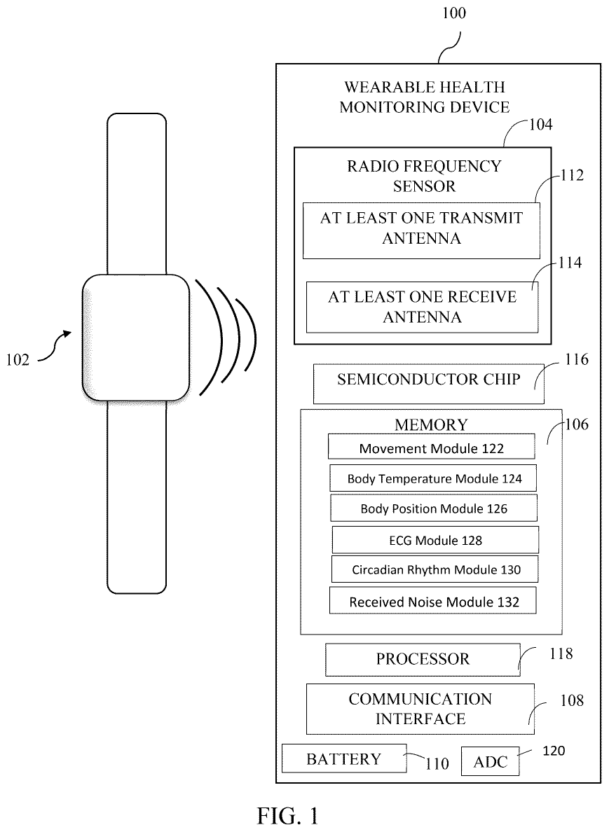

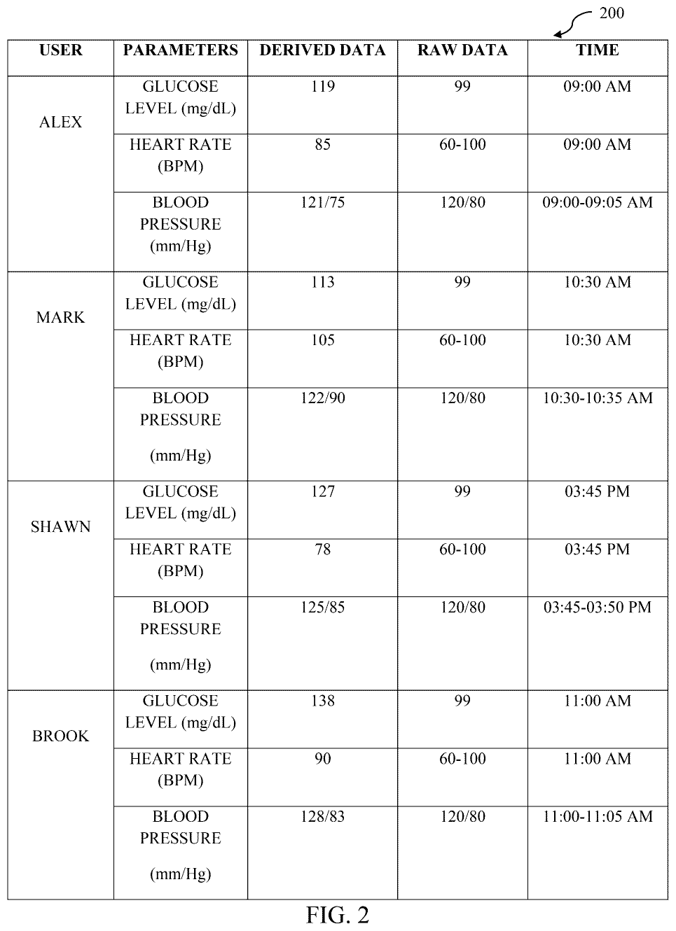

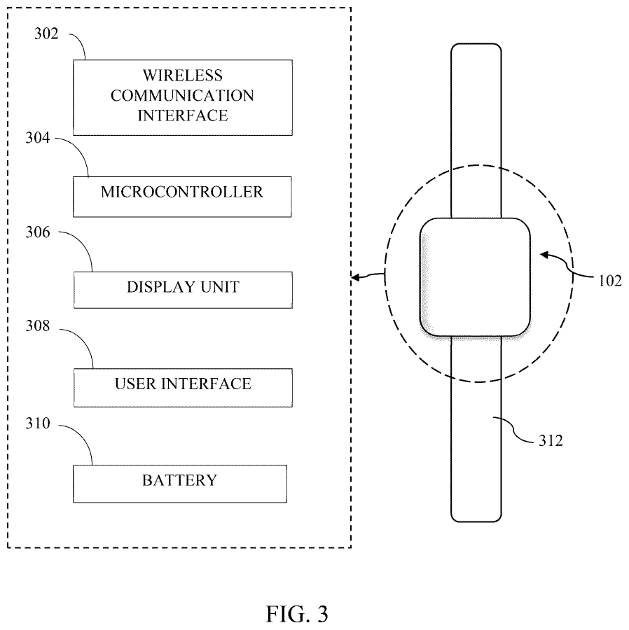

FIG. 1 illustrates a wearable health monitoring device 100 communicatively coupled to a smartwatch 102, according to an embodiment. FIG. 1 is described in conjunction with FIGS. 2-3.

The wearable health monitoring device 100 may comprise a radio frequency sensor 104, memory 106, a communication interface 108, a battery 110, a processor 118, and an analog to digital converter 120. In one embodiment, the wearable health monitoring device 100 may be referred to as an added piece integrated with the smartwatch 102. The wearable health monitoring device 100 may be placed over a body part of a user to determine different health parameters. In one embodiment, the different health parameters may include, but are not limited to, blood glucose level (mg/dL), heart rate (BPM), or blood pressure (mmHg) and SpO2. In one alternate embodiment, the wearable health monitoring device 100 may be communicatively coupled to multiple devices, including a smart phone, a fitness band, a display unit, touch screen, etc.

In an embodiment, the radio frequency sensor 104 may be configured to transmit and receive radio frequency signals of RF Activated Range (between 500 MHZ and 300 GHZ) range to determine the health parameter of the user equipped with the wearable health monitoring device 100. The radio frequency sensor 104 may comprise at least one transmit antenna 112 and at least one receive antenna 114. A semiconductor chip 116 includes one or more components that may be configured to generate the radio frequency signals in a frequency range of 122-126 GHz. Alternatively, the semiconductor chip 116 may comprise a phase locked loop (PLL) (not shown), a band pass filter (BPF), and a mixer (not shown) fabricated on the semiconductor chip 116. For instance, the semiconductor chip 116 generates an analog signal at a frequency range of 9-11 MHz, and such frequency range is fed to the PLL which generates an analog signal in the range of 3-5 GHz frequency. Further, the 3-5 GHz signal is provided to the band pass filter, which filters the analog signal and passes a signal in the 3-5 GHz range to the mixer.

Further, the semiconductor chip 116 may comprise a frequency synthesizer (not shown) and a frequency multiplier (not shown). The frequency synthesizer may be configured to generate multiple output frequencies as multiple of radio frequency signals. Further, the frequency multiplier may be configured to generate an output frequency that is an odd or even multiple of its input frequency. In one embodiment, the semiconductor chip 116 may comprise a frequency mixer (not shown) configured to create new frequencies with respect to applied frequencies.

For example, the frequency synthesizer uses the 9-11 MHz signal to produce a 16 GHz signal. The 16 GHz signal is fed to the frequency multiplier to generate a signal at 120 GHz by doubling the frequency. Further, the produced 3-5 GHz signals and the 120 GHz signals are mixed to generate a signal at 122-120 GHz depending on the frequency between the 3-5 GHz signal. Further, the signals are amplified in the range of 122-128 GHz, these signals are transmitted under the skin surface of the user by the at least one transmit antenna 112.

Further, the RF signals are responded from the user and received by the at least one receive antenna 114 in a form of electromagnetic energy which may be further converted into electrical signals. For example, the electromagnetic energy in the 122-128 GHz frequency band is received by the at least one receive antenna 114 and converted to a 122-128 GHz electrical signal. The semiconductor chip 116 is configured to amplify the 122-128 GHz electrical signal into an amplified output signal in the 122-128 GHz frequency range. The amplified 122-128 GHz signal is mixed with the 128 GHz signal from the frequency multiplier with the received 122-128 GHz signal to generate a 3-5 GHz signal that corresponds to the electromagnetic energy that was received at the at least one receive antenna 114. The 3-5 GHz signal is then mixed with the 3-5 GHz+2.5 MHz signal to generate a 2.5 MHz signal that corresponds to the electromagnetic energy that was received by the at least one receive antenna 114. Further, the semiconductor chip 116 uses the 2 GHz signal and mixes with the 2 GHz+2.5 MHz signals to generate a 2.5 MHz signal. Successively, the 2.5 MHz signal that corresponds to the electromagnetic energy is converted from an analog signal to a digital signal.

In one embodiment, the semiconductor chip 116 may comprise a decimation filter (not shown) configured to reduce sampling frequency of the received signal. It can be noted that the decimation filter may be integrated with an analog to digital converter (ADC) within the semiconductor chip 116. Further, the decimation filter may generate the digital data that represents the electromagnetic energy received by the at least one receive antenna 114. Alternatively, signal processing techniques may be used to achieve beamforming, Doppler effect processing, and/or leakage mitigation in order to separate a desired signal from other undesirable signals. The digital signal processing of incoming signals may use Kalman filters to smooth out noisy data. In one aspect, the digital signal processing of received signals may include digitally merging receive chains. In another aspect, multiple digital signal processing techniques may be utilized to achieve beamforming, Doppler effect processing, and range. Digital signal processing can be accomplished in a digital signal processor (DSP).

Further, the memory 106 may be configured to store the digital data processed by the processor 118. The memory 106 may include suitable logic, circuitry, and/or interfaces that may be configured to store a machine code and/or a computer program with at least one code section executable by the processor 118. Examples of implementation of the memory may include, but are not limited to, Random Access Memory (RAM), Read Only Memory (ROM), Hard Disk Drive (HDD), and/or a Secure Digital (SD) card. In an embodiment, the memory 106 may be configured to store digital information processed by the processor 118 in a database, as shown in FIG. 2.

Further, the database may be segregated into a raw data sample and derived data fetched from the user. In one embodiment, the raw data sample may correspond to standard data or a threshold limit for a specific health parameter to be performed. In one embodiment, the derived data may correspond to data calculated or evaluated from a sample. The raw data sample may be used as a training data set as well as a reference point for the wearable health monitoring device 100 to determine accurate health results of the user. It may be noted that the raw data sample may include data sets of different parameters to allow the wearable health monitoring device 100 to fetch the derived data at different conditions. In one embodiment, the raw data samples include data sets specific to gender, age, specific to weather conditions, specific to regions, etc.

In an embodiment, the communication interface 108 may also be integrated with the wearable health monitoring device 100. The communication interface 108 may be configured to transfer the derived data stored within the database to the smartwatch 102. Further, the communication interface 108 may provide a medium through which the wearable health monitoring device 100 may communicate with a cloud network in the network environment, or with another wearable health monitoring device 100. Such communication may facilitate the user to securely save the data in a cloud database and enable access of the derived data from multiple locations and from multiple devices.

The system 100 may include a movement module 122 that includes at least one sensor from the group of an accelerometer, a gyroscope, an inertial movement sensor, or other similar sensor. The movement module may have its own processor or utilize the processor 118 to calculate movement of the user. Motion from the user will change the blood volume in a given portion of their body, and flow rate of blood in their circulatory system. This may cause noise, artifacts, or other errors in the real-time signals received by the receive antenna 114. The movement module 122 may compare the calculated motion to a motion threshold stored in memory 106. For example, the motion threshold could be movement of more than two centimeters in a one second period. The motion threshold could be near zero to ensure the user is stationary when measuring to ensure the least noise in the RF signal data. When calculated motion levels exceeds the motion threshold the movement module 122 may flag the RF signals collected at the time stamp corresponding to the motion as potentially being inaccurate. In some embodiments, the movement module 122 may compare RF signal data to motion data over time to improve the accuracy of the motion threshold. The movement module 122 may alert the user, such as with an audible beep or warning, or a text message or alert to a connected mobile device. The alert would signal to the user that they are moving too much to get an accurate measurement.

The system 100 may include a body temperature module 124 that includes at least one sensor from the group of a thermometer, a platinum resistance thermometer (PRT), a thermistor, a thermocouple, or other similar temperature sensor. The body temperature module 124 may have its own processor or utilize the processor 118 to calculate the temperature of the user or the user's environment. The user's body temperature, the environmental temperature, and the difference between the two will change the blood volume in a given part of their body, and flow rate of blood in their circulatory system. Variations in temperature from normal body temperature or room temperature may cause noise, artifacts, or other errors in the real-time signals received by the receive antenna 114. The body temperature module 124 may compare the measured temperature to a threshold temperature stored in memory 106. For example, the environmental temperature threshold may be set at zero degrees Celsius because low temperatures can cause a temporary narrowing of blood vessels which may increase the user's blood pressure. When the measured temperature exceeds the threshold the body temperature module 124 may flag the RF signals collected at the time stamp corresponding to the temperature as potentially being inaccurate. In some embodiments, the body temperature module 124 may compare RF signal data to temperature data over time to improve the accuracy of the temperature threshold. The body temperature module may alert the user, such as with an audible beep or warning, or a text message or alert to a connected mobile device. The alert would signal to the user that their body temperature, or the environmental temperature is not conducive to getting an accurate measurement.

The system 100 may include a body position module 126 that includes at least one sensor from the group of an accelerometer, a gyroscope, an inertial movement sensor, or other similar sensor The body position module 126 may have its own processor or utilize the processor 118 to estimate the position of the user. The user's body position may change the blood volume in a given part of their body, and flow rate of blood in their circulatory system. This may cause noise, artifacts, or other errors in the real-time signals received by the receive antenna 114. The body position module 126 may compare the estimated position to a body position threshold stored in memory 106. For example, the smartwatch 102 may be on the user's wrist and the body position threshold may be based on the relative position of the user's hand to their heart. When a user's hand is lower than their heart, their blood pressure will increase, with this effect being more pronounced the longer the position is maintained. Conversely, the higher above a user's holds their arm above their heart, the blood pressure in their hand will be lower. The body position threshold may include some minimum amount of time the estimated body position occurs. When the estimated position exceeds the threshold the body position module may flag the RF signals collected at the time stamp corresponding to the body position as potentially being inaccurate. In some embodiments, the body position module may compare RF signal data to motion data over time to improve the accuracy of the body position threshold. The body position data may also be used to estimate variations is parameters such as blood pressure that correspond to the body position data so as to improve the accuracy of the measurements taken when the user in in that position. The body position module 126 may alert the user, such as with an audible beep or warning, or a text message or alert to a connected mobile device. The alert would signal to the user that their body position is not conducive to getting an accurate measurement.

The system 100 may include an ECG module 128 that includes at least one electrocardiogram sensor. The ECG module 128 may have its own processor or utilize the processor 118 to record the electrical signals that correspond with the user's heartbeat. The user's heartbeat will impact blood flow. Measuring the ECG data may allow the received RF data to be associated with peak and minimum cardiac output so as to create a pulse wave form allowing for the estimation of blood volume at a given point in the wave of ECG data. Variations in blood volume may cause noise, artifacts, or other errors in the real-time signals received by the receive antenna 114. The ECG module 128 may compare the measured cardiac data to a threshold stored in memory 106. For example, the threshold may be a pulse above a 160 bpm, as the increased blood flow volume may cause too much noise in the received RF signal data to generate an accurate measure of blood glucose. When the ECG data exceeds the threshold the ECG module 128 may flag the RF signals collected at the time stamp corresponding to the ECG data as potentially being inaccurate. In some embodiments, the ECG module may compare RF signal data to ECG data over time to improve the accuracy of the ECG data threshold or to improve the measurement of glucose at a given point in the cycle between peak and minimum cardiac output. The ECG module 128 may alert the user, such as with an audible beep or warning, or a text message or alert to a connected mobile device. The alert would signal to the user that their heart rate is not conducive to getting an accurate measurement or requires additional medical intervention.

The system 100 may include a circadian rhythm module 130 that includes at least one sensor measuring actigraphy, wrist temperature, light exposure, and heart rate. The circadian rhythm module 130 may have its own processor or utilize the processor 118 to calculate the user's circadian health. Blood pressure follows a circadian rhythm in that it increases on waking in the morning and decreases during sleeping at night. People with poor circadian health will often have higher blood pressure. These variations in blood pressure can noise, artifacts, or other errors or inaccuracies in the real-time signals received by the receive antenna 114. The circadian rhythm module 130 may compare the circadian data in such a way as to determine whether or not to a threshold is met. The threshold data may be stored in memory 106. For example, the threshold may be set as less than 6 hours of sleep in the last 24 hours. When the observed circadian health data exceeds the threshold the circadian rhythm module 130 may flag the RF signals collected at the time stamp corresponding to circadian health as potentially being inaccurate, or as needing an adjustment to account for the expected increase in the user's blood pressure. In some embodiments, the circadian rhythm module 130 may compare RF signal data to sleep data over time to improve the accuracy of the circadian rhythm thresholds. The circadian rhythm module 130 may alert the user, such as with an audible beep or warning, or a text message or alert to a connected mobile device. The alert would signal to the user that their recent sleep patterns are not conducive to getting an accurate measurement.

The system 100 may include a received noise module 132 that includes at least one sensor measuring background signals such as RF signals, Wi-Fi, and other electromagnetic signals that could interfere with the signals received by the receive antenna 114. The received noise module 132 may have its own processor or utilize the processor 118 to calculate the level of background noise being received. Background noise may interfere with or cause noise, artifacts, or other errors or inaccuracies in the real-time signals received by the receive antenna 114. The received noise module 132 may compare the level and type of background noise to a threshold stored in memory 106. The threshold may be in terms of field strength (volts per meter, and ampere per meter) or power density (watts per square meter). For example, the threshold may be RF radiation at greater than 300 μW/m2. When the background noise data exceeds the threshold the received noise module 132 may flag the RF signals collected at the time stamp corresponding to background noise levels as potentially being inaccurate. In some embodiments, the received noise module may compare RF signal data to background noise over time to improve the accuracy of the noise thresholds. The received radiation module may alert the user, such as with an audible beep or warning, or a text message or alert to a connected mobile device. The alert would signal to the user that the current level of background noise is not conducive to getting an accurate measurement.

Examples of the communication network may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). The communication interface 108 may connect to the communication network via various wired and wireless communication protocols. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols. In an embodiment, the wearable health monitoring device 100 may also be integrated with the battery 110 that may be configured to power all the elements of the device 100.

FIG. 2 illustrates a table 200 representing derived data stored within the database, according to an embodiment.

The database may include multiple health parameters evaluated or derived from one or more users at periodic intervals. The periodic intervals may be set by the one or more users. For example, Alex sets the wearable health monitoring device 100 to measure blood glucose level (in mg/dL) every one-hour to track his diet. It can be noted that the raw sample data may correspond to the threshold limit for each health parameter. In an embodiment, the health parameters may include blood glucose level (mg/dl), heart rate (BPM) and blood pressure (mmHg). In one exemplary embodiment, Alex measures his health parameters at 9:00 AM using the wearable health monitoring device 100, the blood glucose level is 119 mg/dL which is greater than a threshold limit of 99 mg/dL, heart rate is 85 BPM and blood pressure is 121/75 mmHg. In another exemplary embodiment, Mark measures his health parameters while playing baseball using the wearable health monitoring device 100, the blood glucose level is shown as 113 mg/dL which is slightly greater than the threshold limit of 99 mg/dL, heart rate is shown as 105 BPM which is slightly elevated from the threshold limit of 100 BPM and blood pressure is shown as 122/90 mmHg which is slightly greater than the threshold limit of 120/80.

FIG. 3 illustrates a block diagram of components of the smartwatch 102, according to an embodiment.

The smartwatch 102 may include a wireless communication interface 302, a microcontroller 304, a display unit 306, a user interface 308, a battery 310 and a strap 312. In an embodiment, the smartwatch 102 may be wirelessly connected to the wearable health monitoring device 100 via the wireless communication interface 302. The wireless communication interface 302 may receive the derived data stored within the database as well as receive data from the wearable health monitor for processing and display.

Further, the microcontroller 304 may fetch data and display data on the display unit 306. In one embodiment, the display unit 306 may be installed with the user interface 308 that provides an interface to represent the derived data stored within the database. In one exemplary embodiment, the derived data may be represented on the user interface 308 in graphical form, tabular form, text form, etc. Further, the microcontroller 304 is integrated within the smartwatch 102 to facilitate the operation of the smartwatch 102.

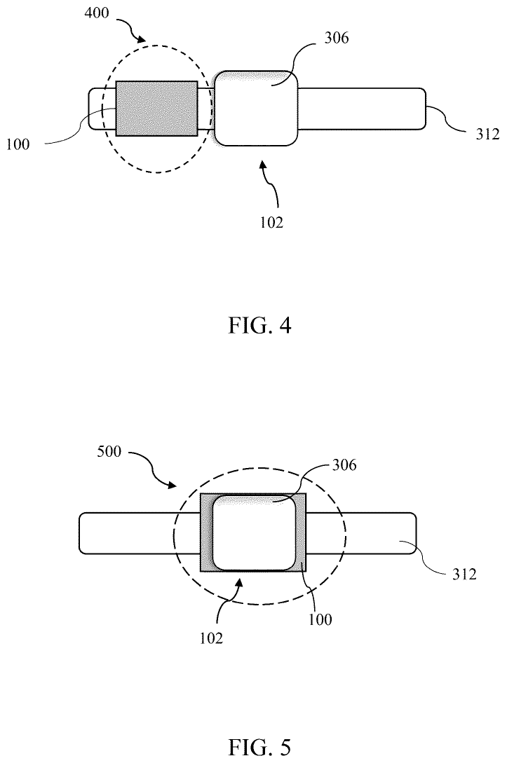

FIG. 4 illustrates the wearable health monitoring device 100 connected to the smartwatch 102 in a clip-on configuration 400, according to an embodiment.

The wearable health monitoring device 100 may be coupled to the strap 312 of the smartwatch 102. The wearable health monitoring device 100 may be attached over the strap 312 via one or more clips (not shown) or one or more magnets (not shown). It may be noted that the wearable health monitoring device 100 may be connected to the smartwatch 102 in any wired or wireless connection options. In one case, the wearable health monitoring device 100 may be connected to the smartwatch 102 via a connection cable (not shown). The connection cable may be connected to the smartwatch 102 and to the wearable health monitoring device 100 through a fabricated port (not shown). In the wireless connection, the wearable health monitoring device 100 may be connected to the smartwatch 102 via the communication interface 108. In one embodiment, the wearable health monitoring device 100 may be fastened to the strap 312 using different fastening means, such as hook and loop fasteners, buckle and hook fasteners, etc.

In one embodiment, the wearable health monitoring device 100 may also be configured with an alignment module (not shown). The alignment module may enable the user to accurately place the wearable health monitoring device 100 over the strap 312 of the smartwatch 102 at a correct orientation. Such orientation of the wearable health monitoring device 100 allows accurate tracking of the health parameters from a specific body location of the user. In one exemplary embodiment, the alignment module includes multiple alignment options such as, but not limited to, visual indicator detection option, radio wave detection option, light ray detection option, and sound wave detection option.

The visual indicator detection option may enable the user to align the wearable health monitoring device 100 by visually judging a center line (not shown) marked over the wearable health monitoring device 100. The center line may be marked over the wearable health monitoring device 100, passing in between the at least one transmit antenna 112 and the at least one receive antenna 114. It can be noted that the center line may correspond to a location of a vein, and the user may simply place the center line over the vein from where the health monitoring may be carried out. Further, the radio wave detection option may enable the user to align the wearable health monitoring device 100 using radio wave signals transmitted by the at least one transmit antenna 112. The at least one transmit antenna 112 may emit radio signals through the skin of the user at various frequencies that may detect the presence of the vein of the user. The at least one receive antenna 114 may be able to determine the presence of the vein of the user and may display over the display unit 306 of the smartwatch 102.

A light ray detection option and a sound wave detection option may also work in a similar manner as the radio wave detection option. In the light ray detection option, a light source (not shown) and a light detector (not shown) may be integrated within the wearable health monitoring device 100. The light source may emit light of a specified wavelength that may penetrate underneath the skin of the user. The light rays of the specified wavelength may reflect from the veins of the user and may be detected by the light detector. Further, the responded light rays may further be analyzed with the help of the processing unit 118 to facilitate accurate alignment of the wearable health monitoring device 100. In the sound wave detection option, the wearable health monitoring device 100 may be integrated with a sound source (not shown) and a sound receiver (not shown). The sound source may transmit sound waves of a specific wavelength that may reflect from the vein of the user and thereby may be detected by the sound receiver. It may be noted that, the alignment options may be configured based upon the placement of the wearable health monitoring device 100 with respect to the smartwatch 102.

In one embodiment, the clip-on configuration 400 may provide feasibility to allow the user to attach or detach the wearable health monitoring device 100 from the strap 312. The clip-on configuration 400 may be suitable in medical research and hospitals where the wearable health monitoring device 100 may be equipped to monitor patients constantly. The clip-on configuration 400 may help the doctor swiftly detach the wearable health monitoring device 100 from the strap 312 of one patient and attach it over another strap of another patient. For example, New York Mercy Hospital conducted a general health checkup for Alex at around 08:45 AM. The wearable health monitoring device 100 is wirelessly connected to the smartwatch 102 and is activated to transmit radio frequency signals underneath the skin surface of Alex and receive the responded radio frequency signals at 120 GHz frequency and wavelength of 2.37 mm. At 8:50 AM the display unit 306 of the smartwatch 102 shows blood glucose level, heart rate, and blood pressure of Alex in tabular form as:

| | | |
|---|---|---|
| Blood Glucose level (mg/dL) | 105 | 08:50 AM |
| Heart rate (BPM) | 85 | 08:50 AM |
| Blood Pressure (mmHg) | 121/71 | 08:45-08:50 AM |

FIG. 5 illustrates the wearable health monitoring device 100 connected to the smartwatch 102 in a shell around configuration 500, according to an embodiment.

The wearable health monitoring device 100 may be structured like a case that may be mounted underneath the smartwatch 102. In one embodiment, the wearable health monitoring device 100 may be designed to encase the smartwatch 102 of a standard size. It can be noted that the wearable health monitoring device 100 may have dimensions greater than the smartwatch 102. In an embodiment, the wearable health monitoring device 100 may be integrated with a magnet (not shown) to facilitate attachment of the wearable health monitoring device 100 underneath the smartwatch 102. In an embodiment, the wearable health monitoring device 100 may be constructed with a taper (not shown) to fit the wearable health monitoring device 100 with the smartwatch 102.

In an embodiment, the wearable health monitoring device 100 may be configured with the radio wave detection option and/or the light ray detection option, and/or the sound wave detection option to enable the user to align the wearable health monitoring device 100 by looking at the display unit 306 of the smartwatch 102. It may be noted that the wearable health monitoring device 100 with the shell around configuration 500 may not be configured with the visual indicator detection option as the top surface (not shown) of the wearable health monitoring device 100 may not be visible once installed underneath the smartwatch 102.

Further, the shell around configuration 500 may provide a rigid and fixed attachment of the wearable health monitoring device 100 with the smartwatch 102. In one embodiment, the rigid and fixed attachment of the wearable health monitoring device 100 with the shell around configuration may be provided in a case when the user is in motion. For example, Mark monitors the general health parameter while playing football at 10:30 AM, using the wearable health monitoring device 100 with the shell around configuration. The display unit 306 thereby shows the data in tabular forms, such as:

| | | |
|---|---|---|
| Glucose level (mg/dl) | 101 | 10:30 AM |
| Heart Rate (BPM) | 99 | 10:30 AM |
| Blood Pressure (mmHg) | 122/80 | 10:30-10:35 AM |

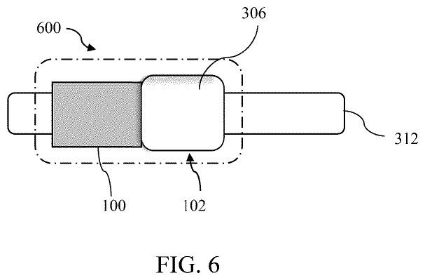

FIG. 6 illustrates the wearable health monitoring device 100 connected to the smartwatch 102 in an interposer configuration 600, according to an embodiment.

The wearable health monitoring device 100 may be directly coupled to the smartwatch 102. The interposer configuration 600 may enable the user to establish a wired connection between the wearable health monitoring device 100 and the smartwatch 102. The wired connection may enable the user to quickly connect or disconnect the wearable health monitoring device 100 with the smartwatch 102. In an embodiment, the direct connection may enable the user to exchange large amount of the data between the wearable health monitoring device 100 and the smartwatch 102. It may be noted that the interposer configuration 600 facilitates a higher data transfer rate in comparison to the wireless connection.

For example, Shawn conducts his general health checkup at 03:45 PM and simultaneously requires sending his previous health data from the memory unit 106 to the smartwatch 102, and the wearable health monitoring device 100 determines the glucose level, heart rate, and blood pressure of Shawn and represent in the form of a table of the display unit 306 as:

| | | |
|---|---|---|
| Glucose level (mg/dl) | 127 | 03:45 PM |
| Heart rate (BPM) | 78 | 03:45 PM |
| Blood Pressure (mmHg) | 125/85 | 03:45-03:50 PM |

For example, Brook is at a party and wears the wearable health monitoring device 102 attached to the smartwatch 102 in any of the prior configurations 400, 500, 600, to check his health parameters. The health parameters are displayed to Brook over the display unit 306 of the smartwatch 102. The displayed information may be projected as:

| | | |
|---|---|---|
| Glucose level (mg/dL) | 113 | 11:00 AM |
| Heart Rate (BPM) | 90 | 11:00 AM |
| Blood Pressure (mmHg) | 128/78 | 11:00-11:05 AM |

Embodiments of the present disclosure will be described more fully hereinafter with reference to the accompanying drawings in which like numerals represent like elements throughout the several figures, and in which example embodiments are shown. However, embodiments of the claims may be embodied in many different forms and should not be construed as limited to the embodiments set forth herein. The examples set forth herein are non-limiting examples and are merely examples among other possible examples.

What is claimed is:

1. A wearable health monitoring device, removeably attachable to a smartwatch having a watch case and a watch strap, comprising:
at least one transmit antenna configured to transmit radio frequency (RF) detection signals into a skin surface of a user;
at least one receive antenna configured to receive responded RF detection signals that result from the RF detection signals transmitted into the user;
an analog to digital converter (ADC) communicatively coupled with the at least one receive antenna, wherein the ADC is configured to convert the received responded RF signals into digital signals;
a processor communicatively coupled to the ADC and configured to convert the digital signals into output information;
a wireless communication module communicatively coupled with the processor and configured to transmit the output information to the smartwatch to reflect a health parameter of the user; and
a structure for physically interfacing with the smartwatch.

2. The wearable health monitoring device of claim 1, further comprising a semiconductor chip including a frequency synthesizer and a frequency multiplier.

3. The wearable health monitoring device of claim 1, further comprising a semiconductor chip including a frequency mixer configured to create new frequencies with respect to applied frequencies.

4. The wearable health monitoring device of claim 1, further comprising a memory configured to store the output information from the processor in a database, the database segregated into a raw data sample and derived data from a user.

5. The wearable health monitoring device of claim 4, wherein the raw data sample may correspond to a threshold limit for each heath parameter.

6. The wearable health monitoring device of claim 1, wherein the database includes multiple health parameters evaluated or derived from one or more users at periodic intervals, where the periodic intervals may be set by the one or more users.

7. The wearable health monitoring device of claim 6, wherein the multiple health parameters include one or more of the following: blood glucose level, heart rate, and blood pressure.

8. The wearable health monitoring device of claim 1, wherein the structure for physically interfacing with the smartwatch includes at least one of: one or more clips that attach over the watch strap, and one or more magnets that attach over the watch strap.

9. The wearable health monitoring device of claim 8, wherein the one or more clips are hook and loop fasteners or buckle and loop fasteners.

10. The wearable health monitoring device of claim 1, wherein the structure for physically interfacing with the smartwatch includes a case that mounts underneath the smartwatch and provides a shell around configuration.

11. The wearable health monitoring device of claim 1, further comprising at least one of:
a motion sensor that detects motion of the user during transmission of the RF detection signals by the one or more transmit antennas and during detection of the RF signals by the one or more receive antennas; and
a body position sensor that detects the body position of the user during transmission of the RF detection signals by the one or more transmit antennas and during detection of the RF signals by the one or more receive antennas;

12. The wearable health monitoring device of claim 1, further comprising at least one of:
a body temperature sensor that detects the temperature of the user during transmission of the RF detection signals by the one or more transmit antennas and during detection of the RF signals by the one or more receive antennas;
an ECG sensor that senses the heartbeat of the user during transmission of the RF detection signals by the one or more transmit antennas and during detection of the RF signals by the one or more receive antennas;
an circadian rhythm sensor that senses circadian data of the user during transmission of the RF detection signals by the one or more transmit antennas and during detection of the RF signals by the one or more receive antennas; and
an ECG background noise sensor that senses background noise around the user during transmission of the RF detection signals by the one or more transmit antennas and during detection of the RF signals by the one or more receive antennas.

13. The wearable health monitoring device of claim 1, further comprising a center line indicator on the wearable health monitoring device disposed in between the at least one transmit antenna and the at least one receive antenna, by which the wearable health monitoring device is properly aligned over the watch strap and vein locations are monitored.

14. The wearable health monitoring device of claim 1, further comprising:
an alignment light source configured to emit light of a specified wavelength into the skin surface of a user, and
an alignment light detector configured to receive reflected light from a vein of the user that results from light emitted into the skin surface of the user, the processor equipped to analyze the reflected light to facilitate alignment of the wearable health monitoring device.

15. The wearable health monitoring device of claim 1, further comprising:
an alignment sound source configured to emit sound waves of a specified wavelength into the skin surface of a user, and
an alignment sound receiver configured to receive reflected sound waves from a vein of the user that results from sound waves emitted into the skin surface of the user, the processor equipped to analyze the reflected sound waves to facilitate alignment of the wearable health monitoring device.

16. A wearable health monitoring device, removeably attachable to a smartwatch having a watch case and a watch strap, comprising:
at least one transmit antenna configured to transmit radio frequency (RF) detection signals into a skin surface of a user;
at least one receive antenna configured to receive responded RF detection signals that result from the RF detection signals transmitted into the user;
an analog to digital converter (ADC) communicatively coupled with the at least one receive antenna, wherein the ADC is configured to convert the received responded RF signals into digital signals;
a processor communicatively coupled to the ADC and configured to convert the digital signals into output information;
one or more clips configured to attach over the watch strap; and
a motion sensor that detects motion of the user during transmission of the RF detection signals by the one or more transmit antennas and during detection of the RF signals by the one or more receive antennas.

17. The wearable health monitoring device of claim 16, further comprising:
an alignment light source configured to emit light of a specified wavelength into the skin surface of a user, and
an alignment light detector configured to receive reflected light from a vein of the user that results from light emitted into the skin surface of the user, the processor equipped to analyze the reflected light to facilitate alignment of the wearable health monitoring device.

18. The wearable health monitoring device of claim 16, further comprising:
an alignment sound source configured to emit sound waves of a specified wavelength into the skin surface of a user, and
an alignment sound receiver configured to receive reflected sound waves from a vein of the user that results from sound waves emitted into the skin surface of the user, the processor equipped to analyze the reflected sound waves to facilitate alignment of the wearable health monitoring device.

19. The wearable health monitoring device of claim 16, further comprising a memory configured to store the output information from the processor in a database, the database segregated into a raw data sample and derived data from a user.

20. The wearable health monitoring device of claim 19, wherein the raw data sample may correspond to a threshold limit for each heath parameter.
